(19) United States
(12) Patent Application Publication
(10) Pub. No.: US 2016/0319231 A1
(43) Pub. Date: Nov. 3, 2016

(54) SOLAR ENERGY FUNNELING USING THERMOPLASTICS FOR ALGAE AND CYANOBACTERIA GROWTH

(71) Applicant: SABIC GLOBAL TECHNOLOGIES B.V., Amsterdam (NL)

(72) Inventors: Suresh Velate, Bangalore (IN); Ihab Nizar Odeh, Sugar Land, TX (US); Duncan Rowe, Thuwal (SA)

(21) Appl. No.: 15/109,944

(22) PCT Filed: Jan. 6, 2015
(86) PCT No.: PCT/US2015/010263

§ 371 Date: Jul. 6, 2016

Related U.S. Application Data
(60) Provisional application No. 61/924,561, filed on Jan. 7, 2014.

(51) Int. Cl. C12M 1/00

(52) U.S. Cl. CPC C12M 21/02; C12M 23/22; C12M 23/20

(57) ABSTRACT

Disclosed is a wavelength conversion material for use in a photo-bioreactor for growing phototrophic organisms. The wavelength conversion material includes an organic fluorescent dye and a polymeric matrix, wherein the organic fluorescent dye is solubilized in the polymeric matrix. The wavelength-conversion material is capable of absorbing light comprising a wavelength of 280 to 650 nm and emitting the absorbed light at a wavelength of 400 to 800 nm.

### CROSS REFERENCE TO RELATED APPLICATIONS

This application claims the benefit to U.S. Provisional Patent Application No. 61/924,561 titled “SOLAR ENERGY FUNNELING USING THERMOPLASTICS FOR ALGAE AND CYANOBACTERIA GROWTH” filed Jan. 7, 2014. The entire contents of the referenced patent application are incorporated into the present application by reference.

### BACKGROUND OF THE INVENTION

A. Field of the Invention

The invention generally concerns wavelength conversion materials for use in a photo-bioreactor for growing phototrophic organisms. The materials include organic fluorescent dyes or combinations of such dyes that are solubilized within a polymeric matrix, where the polymeric matrix is capable of absorbing light from the sun or artificial light and emitting the absorbed light at a wavelength that is beneficial for the growth of the phototrophic organisms (e.g., light having a wavelength of 400 to 800 nm). The phototrophic organisms can then be used to produce bio-fuels and other desired products.

B. Description of Related Art

The largest fraction of the world's energy demand is presently met by the combustion of fossil fuels. This can result in excessive carbon dioxide emissions. Further, fossil fuels are a depleting resource.

Renewable energy sources such as biofuel are a potential alternative to fossil fuels. Biofuels can be derived from various food crops like palm, soy, etc. However, such food crops are limited by feedstock supply (S. Torkamani, Appl. Phys. Lett. 97, 043703 (2010); F. T. Haxo, The Journal of General Physiology 1950, 389). Another alternative is algae fuels. During photosynthesis, algae and other photosynthetic organisms capture carbon dioxide and sunlight and convert it into oxygen and biomass. The biomass can be converted into biofuels such as bioalcohols, biodiesels, biogas, syngas, solid biofuels, etc. by well-known processes (e.g., fermentation and enzymes, thermo/chemical conversion, gasification, etc.). While biomass from algae can have a relatively high energy yield (Lothar Wondraczek, Nature, 2013, Nature, 466 799 (2010)), the efficiency of producing the biomass from phototrophic organisms such as algae and cyanobacteria is relatively low.

Phototrophic organisms rely on light within the Photosynthetically Active Radiation (PAR) region as a source of energy for photosynthesis. The PAR region is typical referred to as light or radiation having a wavelength between 400 to 700 nanometers. Of this, light having a wavelength of about 400 to 500 nanometers (or blue light) and 600 to 700 nanometers (or red light) are more efficiently used by such plants in the photosynthesis process. By comparison, light having a wavelength between about 500 to 600 nanometers (or green/yellow light) is not as efficiently used. Further, light having a wavelength of about 700 to 800 nanometers (or far-red light) promotes petiole elongation while inhibiting germination and rooting in plants. Even further, increasing the red to far-red light (“Red to Far-Red Ratio” or “R:FR”) that a plant receives can be beneficial to the plant's growth and quality.

Attempts have been made to positively impact the growth of phototrophic organisms by manipulating the type and amount of light that said organisms receives. Such attempts typically involve the use of greenhouse materials infused with pigments that are designed to manipulate natural sunlight passing through the materials. In particular, pigments can absorb certain light and reflect others but are inefficient at converting light from one wavelength to another. Other strategies attempt to increase the transmittance of the more important light ranges from 400 to 500 nm or 600 to 700 nm, while simultaneously reflecting the least important range of 500 to 600 nm (see U.S. Pat. No. 4,529,269). The goal of such a strategy is to simply focus on the growth important light and ignore the remaining “unimportant light.”

Problems associated with the current materials are at least three-fold. First, several of the compounds and pigments used are not sufficiently stable from either a photo or thermal stability perspective. This is problematic given that greenhouse materials are typically subjected to prolonged outdoor use. Second, pigments are insoluble particles and have a tendency to coalesce together, which can create uneven distribution into a given material, thereby negatively affecting the efficacy of the material (e.g., some portions of the material may not have pigments or an insufficient amount to perform the desired result). Further, their insolubility limits the amount that can be used in a given material. Third, current strategies do not efficiently modify the R:FR ratio. Either several different types of ingredients are typically used to achieve an acceptable ratio or the strategies appear to be limited to the amount of red/far red light present in natural sunlight.

### SUMMARY OF THE INVENTION

It has been discovered that certain organic fluorescent dyes can be used to produce wave-length conversion materials for use in photo-bioreactors for improving or maximizing the growth of phototrophic organisms. This discovery provides several solutions to the problems existing with the current state of the art in this field. For one, instead of discarding or ignoring the undesirable green/yellow light, it actually converts such light to a more useable red-light. This has the benefit of directly increasing the R:FR ratio without the need for other ingredients. It creates a new source of the desirable red light. Also, the fluorescent dyes have photo and thermal stability, thereby allowing their use in such photo-bioreactors. Further, and unlike pigments, the dyes can be solubilized into a polymeric matrix, which increases the amount of the dyes present within a given matrix and evenly disperses the dyes throughout the matrix. This increased solubility ensures that the resulting polymeric matrix or material provides consistent light converting properties across the entire surface of the material.

In one aspect of the present invention, there is disclosed a wavelength conversion material for use in a photo-bioreactor for growing phototrophic organisms. The wavelength conversion material can include an organic fluorescent dye or a combination of multiple organic fluorescent dyes and a polymeric matrix, wherein the organic fluorescent dye(s) is/are solubilized in the polymeric matrix. The wavelength-conversion material is capable of absorbing light comprising a wavelength of 280 to 650 nm and emitting the absorbed light at a wavelength of 400 to 800 nm. In particular aspects, the wave-length conversion material is capable of absorbing light comprising a wavelength of 450 to 650 nm and emitting the absorbed light at a wavelength of 550 to 800 nm or is capable of absorbing light comprising a wavelength of 280 to 650 nm and emitting the absorbed light at a wavelength of 400 to 700 nm. The wavelength conversion material can be configured such that it is placed between a light source (e.g., sunlight, artificial light source (e.g., UV lamp), or a combination of sunlight and artificial light source) and a plurality of at least one phototrophic organism. The plurality of the at least one phototrophic organism can be included in a liquid medium such as one that includes water. The wavelength-conversion material can be configured to form at least a portion of a container that is configured to hold the liquid medium comprising the plurality of the at least one phototrophic organism. Alternatively, the wavelength conversion material can be a thin sheet that is placed over or adjacent to the container or surrounds the container. In another embodiment, the phototrophic organism can be supported by a substrate (e.g., solid substrate, semi-solid substrate such as a gel substrate, etc.) and a biofilm can be formed by the phototrophic organism. The biomass or biofilm can be formed by subjecting the phototropic organism to light that has been converted by the wavelength conversion material. The phototrophic organisms can be algae (e.g., green algae, red algae, brown algae, golden algae, etc.), other protists (such as euglena), phytoplankton, bacteria (such as cyanobacteria), or combinations thereof. The wavelength-conversion material can be transparent, translucent, or opaque. In particular aspects, it is either transparent or translucent. The polymeric matrix can be formed into a film or a sheet. The film or sheet can be a single-layered or multi-layered film. The film or sheet can be adhered to another surface (e.g., a window, a second film, etc.). The film or sheet can have a thickness of 10 to 500 μm or from 0.5 to 3 mm. The organic fluorescent dyes, polymeric matrix, and/or wavelength-conversion material can have a stoke shift of 60 to 120 nanometers. The polymeric matrix or wavelength-conversion material is thermally stable at a temperature from 200 to 350° C. In particular embodiments, the organic fluorescent dye can be a perylene containing compound, non-limiting examples of which are provided throughout this specification and incorporated into this section by reference. The perylene containing compound can be a perylene diimide. The perylene diimide can have a structure of:

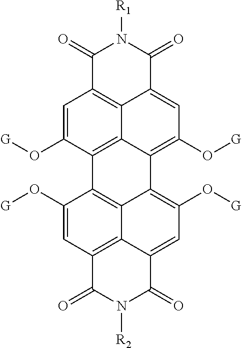

wherein R₁ and R₂ are each independently selected from branched C₆-C₁₈ alkyl and phenyl which is disubstituted by C₁-C₅ alkyl; and G is independently selected from

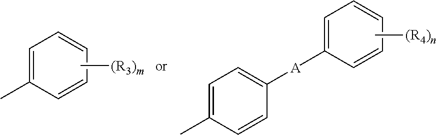

wherein R₃ is independently selected from hydrogen, C₈-C₁₂ alkyl and halogen; m represents the number of substituents and is an integer from 0 to 5; R₄ is independently selected from hydrogen, C₁-C₁₂ alkyl, C₆-C₂₀ aromatic, and C₆-C₂₀ cycloalkyl; n represents the number of substituents and is an integer from 0 to 5; and A is selected from a bond, C₁-C₁₂ alkyl, C₆-C₂₀ aromatic, and C₆-C₂₀ cycloalkyl. Specific non-limiting structures of the perylene dyes are provided in the detailed description and examples section of this specification and are incorporated into this section by reference. In another embodiment, the perylene containing compound can have a structure of:

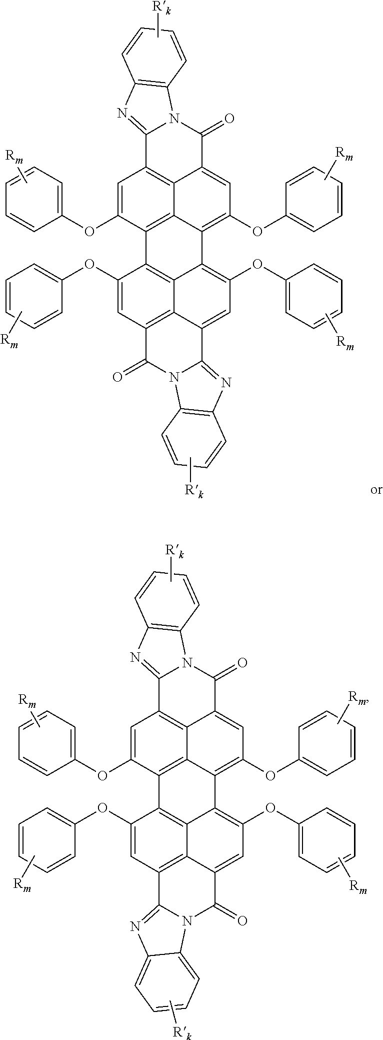

wherein R and R′ are each independently selected from C₈-C₁₈ alkyl, substituted C₈-C₁₈ alkyl, C₈-C₁₈ alkoxy, substituted C₈-C₁₈ alkoxy, and halogen; m represents the number of R substituents on each phenoxy ring, wherein each m is independently an integer from 0 to 5; and k represents the number of R′ substituents on each benzimidazole group, wherein each k is independently an integer from 0 to 4. Specific non-limiting structures of the above perylene compounds are provided in the detailed description and examples section of this specification and are incorporated into this section by reference. In certain aspects, the polymeric matrices of the present invention can include a combination of various perylene compounds. Also, and in addition to the perylene compounds, the organic fluorescent dye can be a coumarin dye, a carbocyanine dye, a phthalocyanine dye, an oxazine dye, a carbostyryl dye, a porphyrin dye, an acridine dye, an anthraquinone dye, an arylmethane dye, a quinone imine dye, a thiazole dye, a bis-benzoxazolylthiophene (BBOT) dye, or a xanthene dye, or any combination of dyes thereof. In certain embodiments, the polymeric matrix can include at least two, three, four, five, six, seven, eight, nine, or ten or more different dyes. In instances where a first and second dye is present in the matrix, the ratio of the first organic fluorescent dye to the second organic fluorescent dye can be from 1:50 to 1:1 to 50:1. The polymeric matrix can include a polycarbonate, a polyolefin, a polymethyl (meth)acrylate, a polyester, an elastomer, a polyvinyl alcohol, a polyvinyl butyral, polystyrene, or a polyvinyl acetate, or any combination thereof. In particular embodiments, the polymeric matrix comprises a polycarbonate or a polyolefin or a combination thereof. Examples of polyolefin polymers include polyethylene or polypropylene polymer. Examples of polyethylene polymers include low-density polyethylene polymers, linear low-density polyethylene polymers, or high-density polyethylene polymers. In some aspects, the polymeric matrix can include an additive. Such additives can be used in a variety of ways (e.g., to increase the structural integrity of the matrix or material, to increase the absorption efficiency of the matrix or material, to aid in dispersing the dyes throughout the matrix, to block ultraviolet rays, infrared rays, etc.). In some instances, the additive can be an ultraviolet absorbing compound, an optical brightener, an ultraviolet stabilizing agent, a heat stabilizer, a diffuser, a mold releasing agent, an antioxidant, an antifogging agent, a clarifier, a nucleating agent, a phosphite or a phosphonite or both, a light stabilizer, a singlet oxygen quencher, a processing aid, an antistatic agent, a filler or a reinforcing material, or any combination thereof. An example of an optical brightener is 2,2′-(2,5-thiophenediyl)bis(5-tert-butylbenzoxazole). In certain aspects, the additive can be a diketopyrrolo-pyrrole (DPP) containing compound. Non-limiting examples of DPP compounds include those having the following structure:

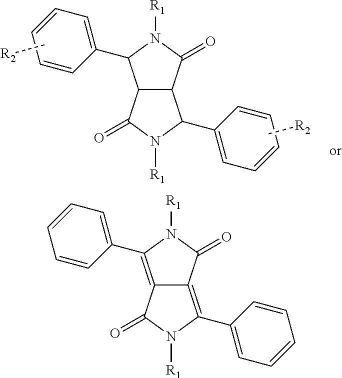

wherein R₁ and R₂ can each individually be H, CH₃, CH₂H₅, 2-ethylhexyl, an amine, or a halogen (e.g., Cl). In particular, embodiments, R₁ and R₂ are each hydrogen. In other instances, R₁ can be hydrogen and R₂ can be a halogen such as Cl. Other derivatives of DPP can also be used in the context of the present invention such that the R₁ and R₂ groups can be C₁ to C₈ linear and branched alkyl groups, phenol groups, etc. In some embodiments, the additive can be a pigment. In other embodiments, the polymeric matrix or wavelength conversion material does not include a pigment or does not include a perylene-based pigment. The polymeric matrix or wavelength converting material can be designed such that it is also capable of absorbing ultraviolet light comprising a wavelength of 280 to 400 nm. In such cases, the polymer matrix can further include an ultraviolet light absorbing compound that is capable of absorbing ultraviolet light comprising a wavelength 280 to 400 nm. In particular instances, the ultraviolet light absorbing compound is capable of emitting said absorbed light in the range of 400 to 800 nm or 400 to 500 nm, or 600 to 700 nm, or 600 to 800 nm. The ultraviolet light absorbing compound can be capable of absorbing ultraviolet light comprising a wavelength of 315 to 400 nm, wherein said compound can be avobenzone (Parsol® 1789, DSM, Switzerland), bisdisulizole disodium (Neo Heliopan® AP, Symrise AG, Germany), diethylamino hydroxybenzoyl hexyl benzoate (Uvinul® A Plus, BASF), ecamsule (Mexoryl™ SX), or methyl anthranilate, or any combination thereof. Avobenzone is also known as methoxydibenzoylmethane and ecamsule is also known as terephthalylidene dicamphor sulfonic acid. The ultraviolet light absorbing compound can be capable of absorbing ultraviolet B light comprising a wavelength of 280 to 315 nm, wherein said compound can be 4-aminobenzoic acid (PABA), cinoxate (2-ethoxyethyl p-methoxycinnamate), ethylhexyl triazone (Uvinul® T 150), homosalate (3,3,5-trimethylcyclohexyl 2-hydroxybenzoate), 4-methylbenzylidene camphor (Parsol® 5000), octyl methoxycinnamate (octinoxate), octyl salicylate (octisalate), padimate 0 (2-ethylhexyl 4-(dimethylamino)benzoate, Escalol® 507, Ashland, Inc.), phenylbenzimidazole sulfonic acid (ensulizole), polysilicone-15 (Parsol® SLX), trolamine salicylate. The ultraviolet light absorbing compound can be capable of absorbing ultraviolet A and B light comprising a wavelength of 280 to 400 nm, wherein said compound can be bemotrizinol (Tinosorb™ S, BASF, USA), benzophenones 1 through 12, dioxybenzone, drometrizole trisiloxane (Mexoryl™ XL), iscotrizinol (Uvasorb® HEB, BASF, USA), octocrylene, oxybenzone (Eusolex® 4360, Merck, KGaA, Germany), or sulisobenzone. The polymeric matrix or wavelength conversion material is capable of emitting more of the absorbed light at a wavelength of 600 to 700 nm than at a wavelength of 700 to 800 nm, thereby increasing the red to far red ratio of the emitted light. In some instances, the polymeric matrix can further include a diffuser such as cross-linked siloxane particles. Non-limiting examples of which include the Tospearl® series diffusers that are commercially available from Momentive Performance Materials, Inc. (e.g., Tospearl® 120, Tospearl® 130, Tospearl® 240, Tospearl® 3120, or Tospearl® 2000.). The diffuser can be an inorganic material comprising antimony, titanium, barium, or zinc, or oxides thereof, and mixtures thereof. In some instances, the organic fluorescent dye is not present on, attached to, or incorporated in silicone flakes or wherein the matrix is not present on, attached to, or incorporated in silicone flakes.

Also disclosed is a photo-bioreactor comprising any one of the wavelength-conversion materials of the present invention. Examples of photo-bioreactors that can be used in combination with the wavelength-conversion materials include plate or flatbed photobioreactors, tubular photobioreactors, bubble column photobioreactors, foil photobioreactors, etc. The wavelength-conversion material can be configured such that it is placed between a light source and a plurality of at least one phototrophic organism. The light source can be natural sunlight or can be artificial (such as from a UV lamp) or can be a combination thereof. The at least one phototrophic organism can be included in a liquid medium that helps promote the growth of the organism (e.g., water can be included in said liquid medium). Alternatively, the organism can be supported by a solid substrate or a semi-solid substrate such as a gel substrate. In either instance, the photo-bioreactor can include a container for holding the liquid medium or a solid or semi-solid substrate for supporting the organism. The photo-bioreactor can be a closed system (e.g., one that includes a transparent or translucent container that encloses the phototropic organism from the environment) or an open system (e.g., an open pond or container that exposes the phototropic organism to the environment). In either instance, the photo-bioreactor can further include a source of carbon dioxide (e.g., a gas inlet that is connected to a source of gas having CO₂). The source can be purified carbon dioxide or can be a mixture of gases, one of which is carbon dioxide. In some aspects, the source of gas can be waste gas such as flue gas. In particular instances, the photo-bioreactor can be a flatbed photo-bioreactor or a column photo-bioreactor. The flatbed photo-bioreactor can include a first surface and an opposing second surface. The opposing second surface can include a reflective backing which can be used to further capture or utilize the light source for growing the phototropic organisms. A second flatbed photo-bioreactor can be placed next to the first surface of the first flatbed photo-bioreactor to form a stack. Third, fourth, fifth, etc. flatbed photo-bioreactors can be placed next to one another to form larger stacks.

Also disclosed is a method of growing a phototrophic organism or biomass comprising obtaining a plurality of at least one phototrophic organism, converting light comprising a wavelength of 280 to 650 nm into light comprising a wavelength of 400 to 800 nm with any one of the wavelength-conversion materials of the present invention, and subjecting the plurality of the at least one phototrophic organism to the converted light. In particular aspects, the method converts light comprising a wavelength of 280 to 400 nm into light comprising a wavelength of greater than 400 to 800 nm or greater than 400 to 700 nm. As discussed above and throughout this specification, the at least one phototrophic organism can be comprised within a liquid medium or can be supported on a substrate (e.g., solid substrate, semi-solid substrate such as a gel substrate, etc.). Further, the method can include the use of a photo-bioreactor system. The light source can be sunlight or artificial light or a combination thereof. The rate of growth of the plurality of the at least one phototrophic organism or biomass can increase when compared with the rate of growth of a plurality of the at least one phototrophic organism or biomass that has not subjected to the converted light. The produced organisms or biomass can then be harvested and further converted into additional products such as biofuels (e.g., bioalcohols, biodiesels, biogas, syngas, solid biofuels, etc.). Well-known processes for making said biofuels can be utilized (e.g., fermentation and enzymes, thermo/chemical conversion, gasification, etc.).

In a further aspect of the invention there is disclosed a method of increasing the red to far red (R:FR) ratio of red light that a phototropic organism receives comprising converting light comprising a wavelength of 500 to 700 nm into light comprising a wavelength of greater than 550 to 800 nm with any one of the wavelength-conversion materials or matrixes discussed above and throughout this specification. The method can further include subjecting the phototropic organism to the converted light, wherein the R:FR ratio of red light that the organism receives is increased by at least 5, 10, 15, 20, 30, 40, or 50% or more in the presence of the converted light when compared with the R:FR ratio of red light that an organism receives in the absence of said converted light. The majority of the converted light can include a wavelength of 600 to 700 nm. In some instances, the R:FR ratio can be measured by using specific absorbances for red and far-red light such as 660 nm for red and 730 nm for far-red. The 660/730 Sensor (Red/Far Red) commercially available from Skye Instruments Ltd. (United Kingdom) can be used. The light source can be natural sunlight or can be non-natural light produced from a light source such as a lamp. As noted above, the wavelength-conversion material can be a film or sheet. The material can be used in a variety of photo-bioreactors, including open and closed-systems.

In another embodiment of the invention there is disclosed a method of making any one of the wave-length conversion materials of the present invention. The method can include obtaining an organic fluorescent dye that is capable of absorbing light comprising a wavelength of 280 to 650 nm and emitting the absorbed light at a wavelength of 400 to 800 nm, and adding said organic fluorescent dye into a polymeric matrix such that said dye is solubilized in said polymeric matrix. The organic fluorescent dye can be added to the polymer matrix to form a mixture. The mixture can be extruded with an extruder to form an extrudate. The extrudate can be formed into a sheet or film or molded into a container. The organic fluorescent dye can be added to the polymer matrix in powdered form or as a solution in which the dye is partially or fully solubilized within a solvent. The extrudate can be molded into a container that is capable of holding a liquid medium comprising a phototrophic organism such as algae or cyanobacteria.

The term “photo-bioreactor” refers to a structure used to grow phototropic organisms. The photo-bioreactors can be closed-systems system (e.g., one that includes a transparent or translucent container that encloses the phototropic organism from the environment) or an open system (e.g., an open pond or container that exposes the phototropic organism to the environment).

The term “integer” means a whole number and includes zero. For example, the expression “n is an integer from 0 to 4” means n may be any whole number from 0 to 4, including 0.

Compounds are described using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, the aldehyde group —CHO is attached through the carbon of the carbonyl group.

The term “aliphatic” refers to a linear or branched array of atoms that is not cyclic and has a valence of at least one. Aliphatic groups are defined to comprise at least one carbon atom. The array of atoms may include heteroatoms such as nitrogen, sulfur, silicon, selenium and oxygen or may be composed exclusively of carbon and hydrogen (“Alkyl”). Aliphatic groups may be substituted or unsubstituted. Examples of aliphatic groups include, but are not limited to, methyl, ethyl, isopropyl, isobutyl, chloromethyl, hydroxymethyl (—CH₂OH), mercaptomethyl (—CH₂SH), methoxy, methoxycarbonyl (CH₃OCO—), nitromethyl (—CH₂NO₂), and thiocarbonyl.

The term “alkyl” refers to a linear or branched array of atoms that is composed exclusively of carbon and hydrogen. The array of atoms may include single bonds, double bonds, or triple bonds (typically referred to as alkane, alkene, or alkyne). Alkyl groups may be substituted or unsubstituted. Examples of alkyl groups include, but are not limited to, methyl, ethyl, and isopropyl.

The term “aromatic” refers to an array of atoms having a valence of at least one and comprising at least one aromatic group. The array of atoms may include heteroatoms such as nitrogen, sulfur, selenium, silicon and oxygen, or may be composed exclusively of carbon and hydrogen. The aromatic group may also include nonaromatic components. For example, a benzyl group is an aromatic group that comprises a phenyl ring (the aromatic component) and a methylene group (the nonaromatic component). Examples of aromatic groups include, but are not limited to, phenyl, pyridyl, furanyl, thienyl, naphthyl, biphenyl, 4-trifluoromethylphenyl, 4-chloromethylphen-1-yl, and 3-trichloromethylphen-1-yl(3-CCl₃Ph—).

The terms “cycloaliphatic” and “cycloalkyl” refer to an array of atoms which is cyclic but which is not aromatic. The cycloaliphatic group may include heteroatoms such as nitrogen, sulfur, selenium, silicon and oxygen, or may be composed exclusively of carbon and hydrogen. A cycloalkyl group is composed exclusively of carbon and hydrogen. A cycloaliphatic group may comprise one or more noncyclic components. For example, a cyclohexylmethyl group (C₆H₁₁CH₂) is a cycloaliphatic functionality, which comprises a cyclohexyl ring (the array of atoms which is cyclic but which is not aromatic) and a methylene group (the noncyclic component). Exemplary cycloaliphatic groups include, but are not limited to, cyclopropyl, cyclobutyl, 1,1,4,4-tetramethylcyclobutyl, piperidinyl, and 2,2,6,6-tetramethylpiperydinyl.

The term “alkoxy” refers to an array of atoms containing an alkyl group and an oxygen atom at one end. Alkyl groups may be substituted or unsubstituted. Examples of alkoxy groups include methoxy(-OCH₃) and ethoxy(-OCH₂CH₃). A related group is “phenoxy,” which refers to a phenyl group having an oxygen atom attached to one carbon. The phenoxy group may also be substituted or unsubstituted.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The wavelength-conversion materials, organic fluorescent dyes, and/or polymeric matrices of the present invention can “comprise,” “consist essentially of” or “consist of” particular ingredients, components, compositions, etc. disclosed throughout the specification. With respect to the transitional phase “consisting essentially of,” in one non-limiting aspect, a basic and novel characteristic of the wavelength-conversion materials, organic fluorescent dyes, and/or polymeric matrices of the present invention are their ability to efficiently absorb light comprising a wavelength of 500 to 700 nm and emitting the absorbed light at a wavelength of greater than 550 to 800 nm.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is an absorption spectrum of prior art single-celled colony forming cyanobacteria.

FIGS. 2A through 2F depict schematics of bioreactors of the present invention.

FIG. 3 is an emission spectrum of perylene-based dyes of the present invention in a low density polyethylene film.

FIG. 4 is a cyanobacteria culture covered with test polyethylene sleeves.

FIG. 5 is a biomass dry weight measured for microalgae and cyanobacteria growth under simulated light conditions.

FIG. 6 depicts a relative spectra of sunlight measured for PE films containing two concentrations of perylene-based dyes of the present invention.

FIG. 7 is cyanobacterium culture in polyethylene bags doped with perylene-based dyes of the present invention.

FIG. 8 is a graph of days of outdoor growth in August of cyanobacterium culture dry weight in grams per liter.

FIG. 9 is a graph of days of outdoor growth in November of a cyanobacterium culture dry weight in grams per liter.

FIG. 10A depicts a transmission electron microscopy (TEM) image of the cyanobacterium cell culture of FIG. 9 grown in a control bag.

FIG. 10B depicts a TEM image of the cyanobacterium cell culture of FIG. 9 grown in a bag doped with 0.12 wt. % of perylene-based dyes of the present invention.

### DETAILED DESCRIPTION OF THE INVENTION

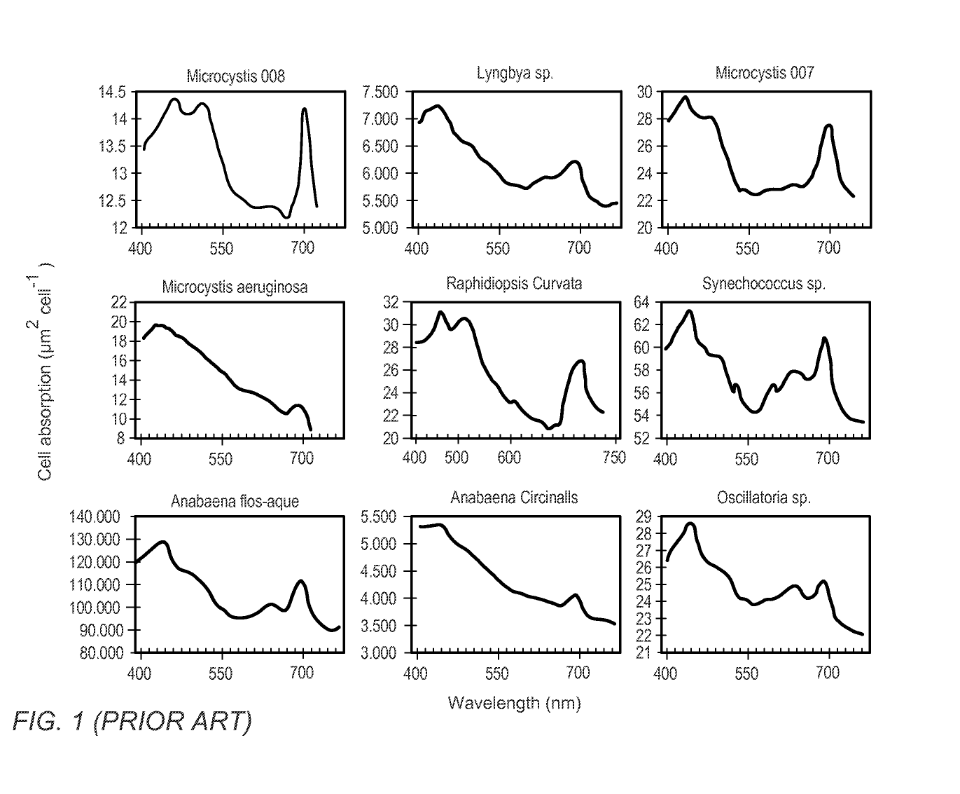

While the use of phototropic organisms such as algae and cyanobacteria as feedstock for biofuel production is known, the yield and quality of producing the feedstock remains largely inefficient. One potential cause of this is illustrated in Table 1 and FIG. 1 (Limol. Oceanogr. 37(2), 1992, 434). For instance, and as illustrated in Table 1, different species of algae display high photosynthetic activity at different spectral frequencies (see Ref: F. T. Haxo 1949):

TABLE 1
(Relative Photosynthetic Rates of Various Algae at Different Wavelengths)

| | 435.8 nm | 546 nm | 620-660 nm |
|---|---|---|---|
| Ulva (Green Algae) | 94% | 46% | 100% |
| Schizymenia (Red Algae) | 48-53% | 288-340% | 100% |

Similarly, the absorbance spectrum of different cyanobacteria (FIG. 1) varies. From these absorbance spectra, it is evident that certain strains of algae and cyanobacteria do not utilize light having a wavelength of 500 to 600 nm efficiently, where sun has its highest intensity.

The present discovery offers a solution to these inefficiencies by manipulating the wavelength of light received by phototropic organisms such as algae and cyanobacteria. It was discovered that certain organic fluorescent dyes can be solubilized in a polymer matrix and used in wavelength-conversion materials to increase the phototropic organisms' use of available light to aid in plant growth. These materials work by absorbing light that the phototropic organisms are inefficient at absorbing and converting said light to light that said organisms can efficiently absorb. In particular, when light falls onto the wavelength-conversion materials, the luminescent dyes absorb light in the spectral region where the phototropic organisms have lesser absorption and emit in the region where they have higher absorption rates. As illustrated in the Examples, this increases the overall flux of harvestable light for the phototropic organisms. Therefore, the wave-length conversion materials of the present invention can increase the efficiency of the photosynthesis process for phototropic organisms such as algae and cyanobacteria.

These and other non-limiting aspects of the present invention are discussed in further detail in the following sections.

### A. Organic Fluorescent Dyes

A variety of organic fluorescent dyes can be used in the context of the present invention. In particular embodiments, perylene-based dyes can be used in the wavelength-conversion material of the present invention. The dyes are capable of absorbing light comprising a wavelength of 500 to 700 nm or 500 to 600 nm and emitting the absorbed light at a wavelength of greater than 550 to 800 nm or 600 to 800 nm or 600 to 700 nm.

Perylene-based organic fluorescent dyes are derived from perylene, which has the following chemical structure:

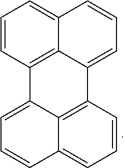 [SMILES: c1cc2cccc3c4cccc5cccc(c(c1)c23)c54]

Non-limiting examples of perylene-based dyes that can be used are described in U.S. Pat. Nos. 8,299,354, 8,304,645, and 8,304,647, the disclosures of which are incorporated by reference.

In one particular instance, the structure of the perylene-based organic fluorescent dye can be a perylene diimide of Formula (I):

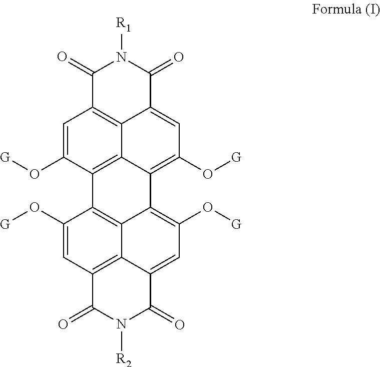

wherein R₁ and R₂ are each independently selected from branched C₆-C₁₈ alkyl and phenyl which is disubstituted by C₁-C₅ alkyl; and G is independently selected from Formulas (Ia) and (Ib):

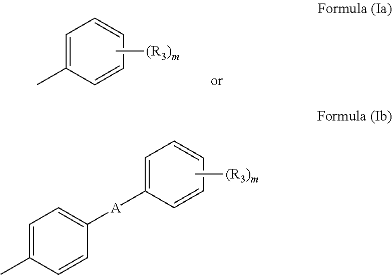

wherein R₃ is independently selected from hydrogen, C₈-C₁₂ alkyl and halogen; m represents the number of substituents and is an integer from 0 to 5; R₄ is independently selected from hydrogen, C₁-C₁₂ alkyl, C₆-C₂₀ aromatic, and C₆-C₂₀ cycloalkyl; n represents the number of substituents and is an integer from 0 to 5; and A is selected from a bond, C₁-C₁₂ alkyl, C₆-C₂₀ aromatic, and C₆-C₂₀ cycloalkyl. In particular embodiments, R₁ and R₂ are independently selected from branched C₆-C₁₈ alkyl; each R₃ is independently selected from C₈-C₁₂ alkyl; and m is an integer from 1 to 5. The four G groups can be the same or different. In one aspect, the G group can be

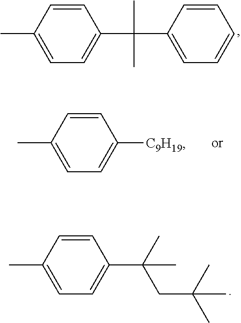

In particular embodiments, the G group is Formula (Ib).

In one aspect, the perylene diimide fluorescent dye has a structure of Formulas (II), (III), (IV), (V), or (VI):

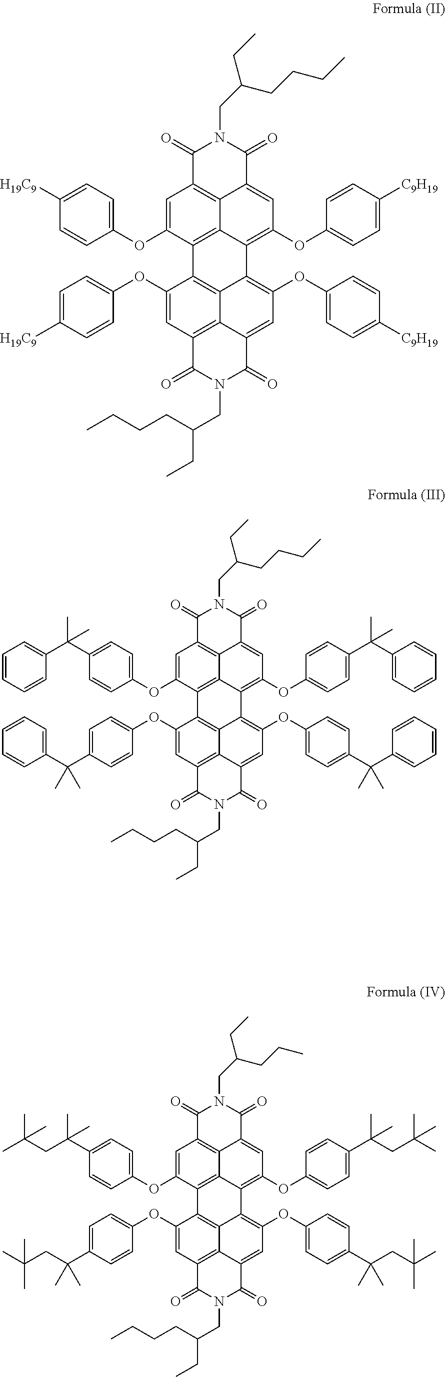

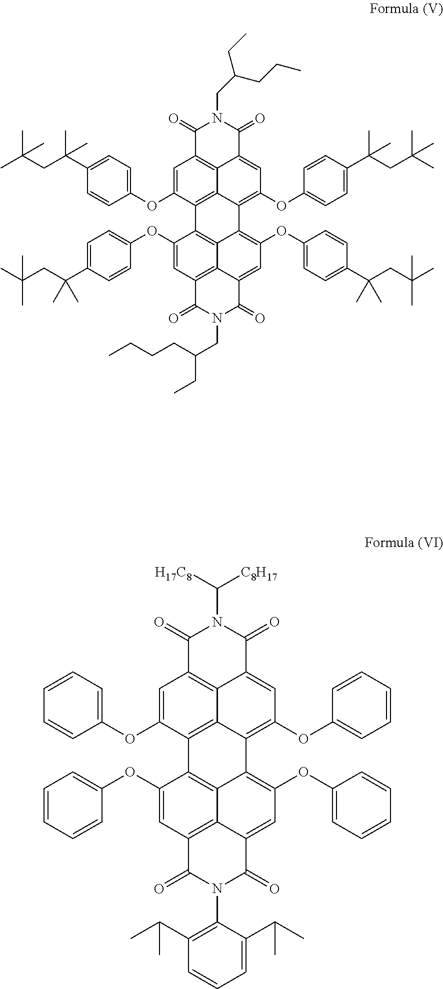

In another aspect, the perylene diimide fluorescent dyes can be based on the Lumogen® series of dyes, which are commercially available from BASF. In one particular instance, the following Lumogen® F Red 305 dye can be used:

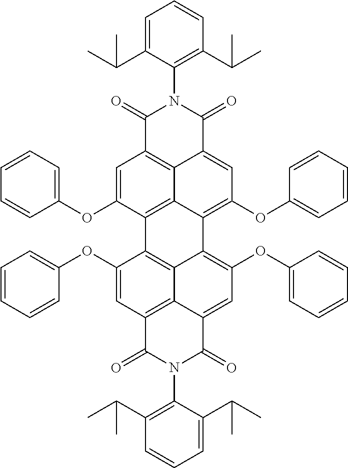 [SMILES: CC(C)c1cccc(C(C)C)c1N1C(=O)c2cc(Oc3ccccc3)c3c4c(Oc5ccccc5)cc5c6c(cc(Oc7ccccc7)c(c7c(Oc8ccccc8)cc(c2c37)C1=O)c64)C(=O)N(c1c(C(C)C)cccc1C(C)C)C5=O]

A method for making the above-noted compounds is provided in U.S. Pat. No. 8,304,647, which again is incorporated by reference. In general, the following reaction scheme can be used:

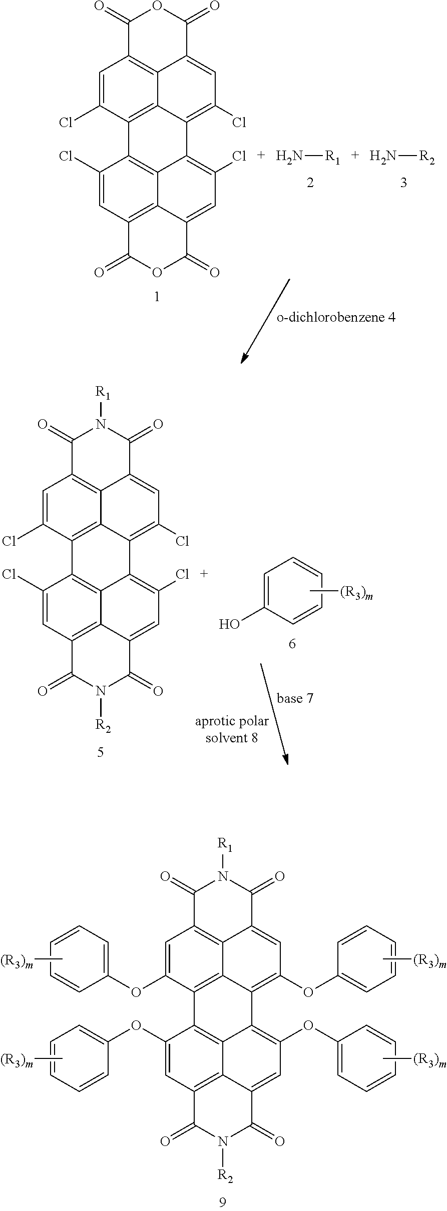

Referring to the above reaction scheme, the method of making a compound of Formula (I) can include condensing a tetrachloroperylene dianhydride 1 with an amine of the formula H₂N—R₁ 2 and an amine of the formula H₂N—R₂ 3 in o-dichlorobenzene 4. If R₁ and R₂ are identical, then the dianhydride is condensed with only one amine. The intermediate product 5 formed from the reaction of the tetrachloroperylene dianhydride and amine(s) can be used without purification or separation if desired. The intermediate product is then reacted with a base 6 and a phenol 7 in an aprotic polar solvent 8 to obtain the dye compound 9 of Formula (I). The phenol reacts with the base to form a phenol salt that more easily reacts with the intermediate product. In specific embodiments, the base is a potassium or sodium base. Examples of bases include potassium carbonate (K₂CO₃), sodium carbonate, and similar bases. Especially desirable are bases having a pKa of 10 or less. The phenol used to react with the intermediate product generally has the structure of Formula (Ic) or (Id):

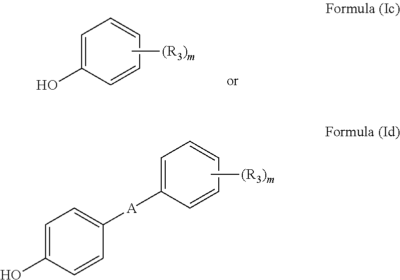

where R₃, m, R₄, n, and A are as described above. Exemplary phenols include nonyl phenol, p-cumyl phenol, and p-tert-octyl phenol. Suitable aprotic polar solvents include dimethylformamide (DMF); n-methyl pyrrolidone (NMP); dimethyl sulfoxide (DMSO); dimethylacetamide, and halogenated solvents like o-dichlorobenzene. The condensing reaction of the tetrachloroperylene dianhydride and amine(s) can be performed at temperatures of from about 80° C. to about 200° C. The condensing reaction may take place over a time period of from about 2 hours to about 10 hours, including from about 4 hours to about 8 hours. The reaction of the intermediate product with the salt and the phenol can be performed at temperatures of from about 80° C. to about 220° C. In more specific embodiments, the temperature is from about 160° C. to about 200° C. The condensing reaction may take place over a time period of from about 30 minutes to about 36 hours. In more specific embodiments, the time period is from about 1 hour to about 28 hours. The reaction of the intermediate product with the base and the phenol may also take place in an inert atmosphere, such as under nitrogen or argon gas. Desirably, the solvent is “dry”, i.e. contains as little water as possible. After the dye compound of Formula (I) is formed, it may be purified by column chromatography. The dye compounds are soluble in common solvents like chlorobenzene, dichlorobenzene, toluene, chloroform, dichloromethane, cyclohexane, and n-hexane.

In another particular instance, the perylene-based organic fluorescent dye (a nonyl phenol containing type lumogen red dye (NRL)) can have a structure of Formula (VII) and (VIII):

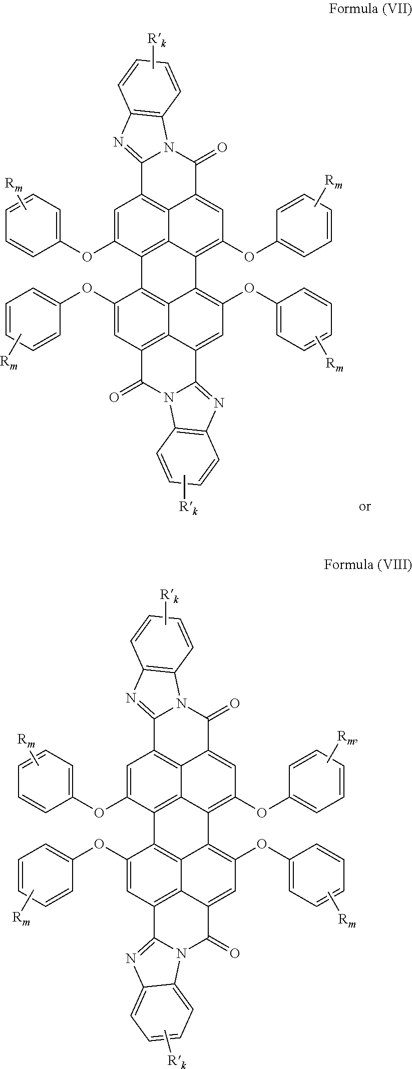

wherein each R and R′ is independently selected from C₁-C₁₈ alkyl, substituted C₁-C₁₈ alkyl, C₁-C₁₈ alkoxy, substituted C₁-C₁₈ alkoxy, and halogen; m represents the number of R substituents on each phenoxy ring, wherein each m is independently an integer from 0 to 5; and k represents the number of R′ substituents on each benzimidazole group, wherein each k is independently an integer from 0 to 4. The compounds can be considered as having a perylene core, two benzimidazole end groups (trans and cis isomers), and four phenoxy side groups. The hydrogen atoms of the alkyl and alkoxy groups may be substituted with, for example, hydroxyl and phenyl groups.

In some specific embodiments, each phenoxy group is substituted in only the para position with an R group independently selected from C₈-C₁₈ alkyl (with respect to the oxygen atom) (i.e. m=1). In more specific embodiments, the four R groups in the para position are the same. In other specific embodiments, each k is zero.

In particular embodiments, each R and R′ is independently selected from C₈-C₁₈ alkyl, substituted C₈-C₁₈ alkyl, C₈-C₁₈ alkoxy, substituted C₈-C₁₈ alkoxy, and halogen; each m is independently an integer from 0 to 5; and each k is independently an integer from 0 to 4.

Non-limiting examples of compounds of Formulas (VII) and (VIII) are provided below in Formulas (IX), (X), (XI), (XII), (XIII), (IVX):

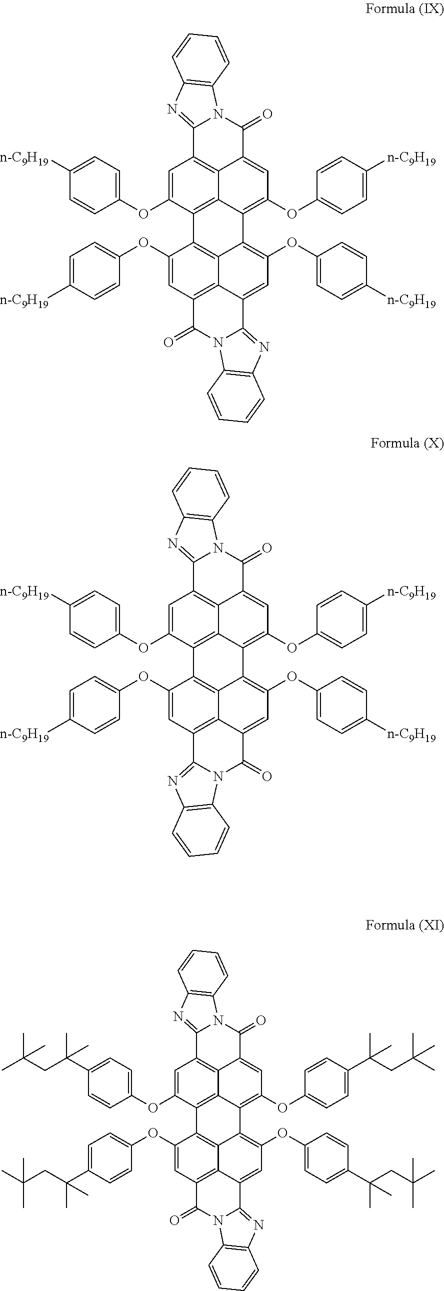

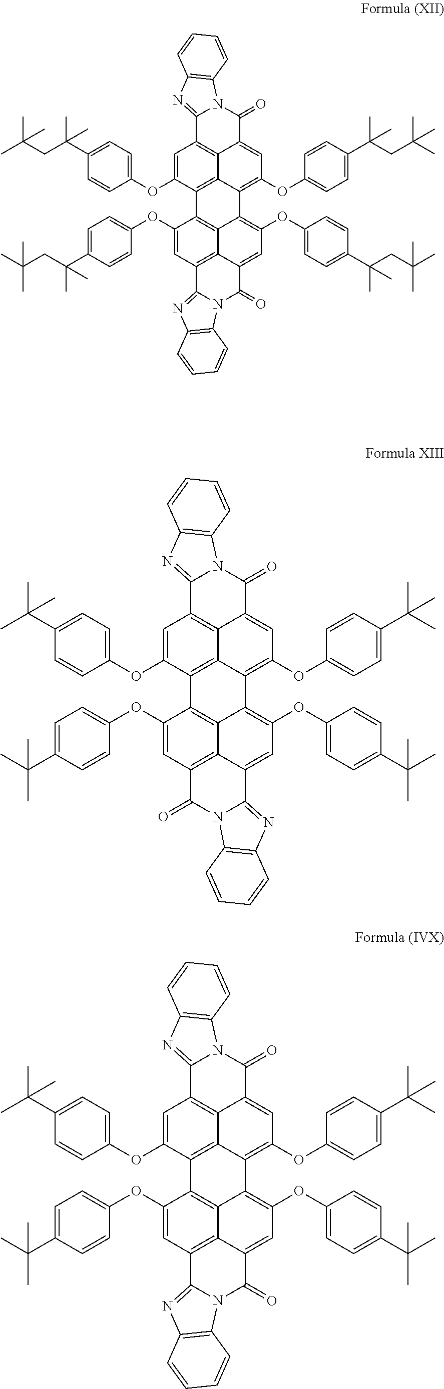

A method for making the above-noted compounds is provided in U.S. Pat. No. 8,299,354, which again is incorporated by reference. In general, the following reaction scheme can be used:

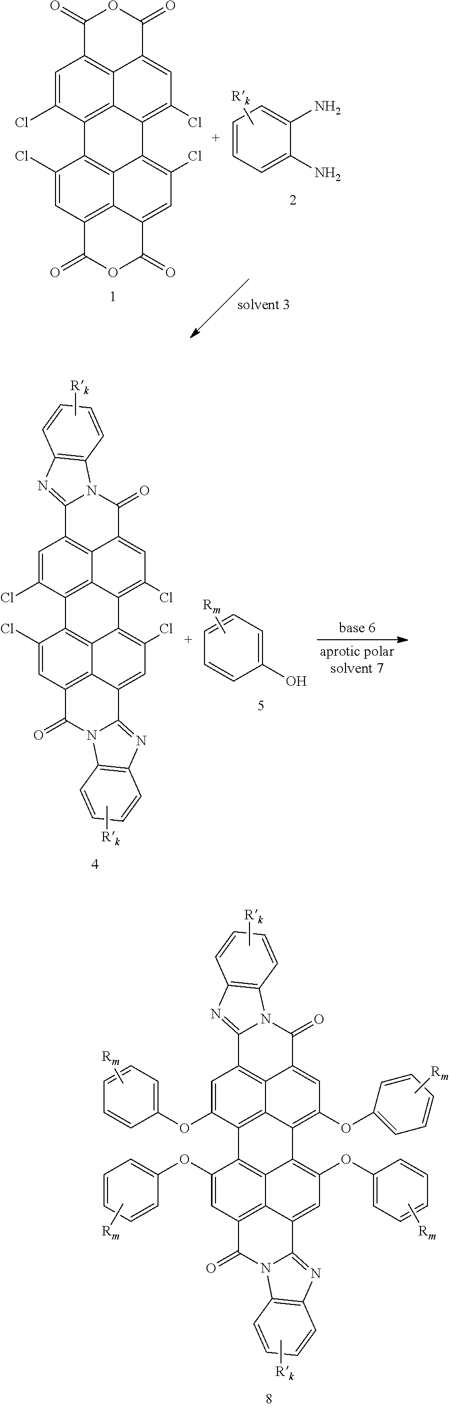

Referring to the above reaction scheme, the dye compounds of Formulas (VII) and (VIII) can be synthesized by condensing a tetrachloroperylene dianhydride 1 with an o-phenylene diamine 2 in an appropriate solvent 3. The intermediate product 4 formed from the reaction of the tetrachloroperylene dianhydride and o-phenylene diamine can be used without purification or separation. The intermediate product is then reacted with a base 5 and a phenol 6 in an aprotic polar solvent 7 to obtain the dye compound 8 of Formula (VII) or (VIII) (here, only Formula (VII) is shown). The o-phenylene diamine (also known as diaminobenzene) is used to form the benzimidazole end groups of the dye compound. If desired, substituted o-phenylene diamines may also be used. The o-phenylene diamines may be substituted with C₁-C₁₈ alkyl, substituted C₁-C₁₈ alkyl, C₁-C₁₈ alkoxy, substituted C₁-C₁₈ alkoxy, and halogen. Appropriate solvents for the condensation of the tetrachloroperylene dianhydride and o-phenylene diamine include propionic acid, acetic acid, imidazole, quinoline, isoquinoline, N-methylpyrrolidone, dimethylformamide, and halogenated solvents like o-dichlorobenzene. The phenol reacts with the base to form a phenol salt that more easily reacts with the intermediate product. In specific embodiments, the base is a potassium or sodium base. Exemplary bases include potassium carbonate (K₂CO₃), sodium carbonate, and similar bases. Especially desirable are bases having a pKa of 10 or less. The phenol used to react with the intermediate product generally has the structure of Formula (XV):

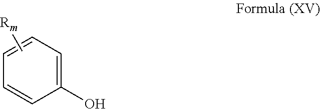

where R and m are as described above. Examples of phenols include nonyl phenol; p-tert-butyl phenol; and p-tert-octyl phenol. Suitable aprotic polar solvents include dimethylformamide (DMF); n-methyl pyrrolidone (NMP); dimethyl sulfoxide (DMSO); dimethylacetamide; and halogenated solvents like o-dichlorobenzene. The condensing reaction of the tetrachloroperylene dianhydride and o-phenylene diamine can be performed at temperatures of from about 80° C. to about 200° C. The condensing reaction may take place over a time period of from about 3 hours to about 12 hours, including from about 4 hours to about 8 hours. The reaction of the intermediate product with the base and the phenol can be performed at temperatures of from about 80° C. to about 200° C. In more specific embodiments, the temperature is from about 130° C. to about 160° C. The condensing reaction may take place over a time period of from about 4 hours to about 36 hours. In more specific embodiments, the time period is from about 4 hours to about 28 hours. The reaction of the intermediate product with the base and the phenol may also take place in an inert atmosphere, such as under nitrogen or argon gas. Desirably, the solvent is “dry”, i.e. contains as little water as possible. After the dye compound of Formula (VII) or (VIII) is formed, it may be purified by column chromatography. The dye compounds are soluble in common solvents like chlorobenzene, dichlorobenzene, toluene, chloroform, and dichloromethane.

In addition to the above-discussed perylene-based dyes, it is also contemplated that other dyes can be used in the context of the present invention. Non-limiting examples of such other dyes include coumarin dyes, carbocyanine dyes, phthalocyanine dyes, oxazine dyes, carbostyryl dyes, porphyrin dyes, acridine dyes, anthraquinone dyes, arylmethane dyes, quinone imine dyes, thiazole dyes, bis-benzoxazolylthiophene (BBOT) dyes, or xanthene dyes, or any combination of such dyes.

### B. Polymer Matrices

The organic fluorescent dyes can be incorporated into a polymeric matrix. One of the advantages of using these dyes is that they can be solubilized within said matrix, thereby providing for a more even distribution of the dyes throughout the matrix when compared with pigments. The polymer matrix/dye combination can be manufactured by methods generally available in the art. For example, the dye compounds of the present invention can be easily incorporated into a wide range of polymers that can be used in photo-bioreactors. Non-limiting examples of such polymers include a polycarbonate, a polyolefin, a polymethyl (meth)acrylate, a polyester, an elastomer, a polyvinyl alcohol, a polyvinyl butyral, polystyrene, or a polyvinyl acetate, or any combination thereof. In particular aspects, the polymeric matrix includes a polycarbonate or a polyolefin or a combination thereof

The fluorescent dyes of the present invention can be added either as a powder or as a solution in a suitable solvent to the polymeric matrix. Generally, the dyes can be distributed within the polymer (e.g., polycarbonate, polyolefin, etc.) by using any means which accomplish the purpose, such as by dispersion. Additives may also be compounded into a masterbatch with a desired polymeric resin and fed into the extruder. The extruder is generally operated at a temperature higher than that necessary to cause the composition to flow. That is, during extrusion the polymer will melt and the dye will solubilize in the polymer composition. The extrudate is immediately quenched in a water bath and pelletized. The pellets, so prepared, when cutting the extrudate may be one-fourth inch long or less as desired. Such pellets may be used for subsequent molding, shaping, or forming. The polymeric matrices may be molded into films, sheets, and other wavelength conversion materials by a variety of means such as injection molding, extrusion, rotational molding, blow molding and thermoforming.

In addition to the organic fluorescent dyes, additives can also be added to the polymeric matrices by the same processes as described above, with a proviso that the additives are selected so as not to adversely affect the desired wavelength conversion properties of the matrices and materials of the present invention. Either a single additive or multiple additives can be used. Such additives may be mixed at a suitable time during the mixing of the components for forming the polymeric matrices of the present invention. Non-limiting examples of additives that may be included in the matrices or materials of the present invention are provided below. The additives can help strengthen the matrices and materials of the present invention, further aid in plant growth, etc. Such additives include, but are not limited to, ultraviolet absorbing compounds, optical brighteners, ultraviolet stabilizing agents, heat stabilizers, diffusers, mold releasing agents, antioxidants, antifogging agents, clarifying agents, nucleating agents, phosphites or phosphonites or both, light stabilizers, singlet oxygen quenchers, processing aids, antistatic agents, fillers or reinforcing materials, or any combination thereof. Each of these additives can be present in amounts of from about 0.0001 to about 10 weight percent, based on the total weight of the polymeric matrix or materials of the present invention.

### C. Uses of the Wavelength Conversion Materials in Photo-Bioreactors

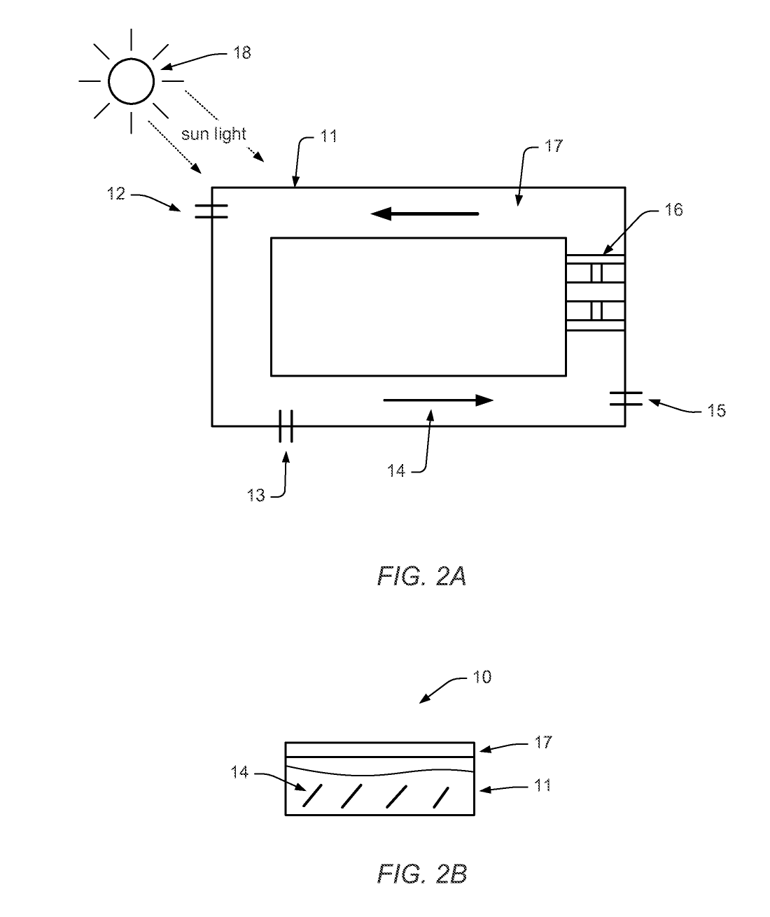

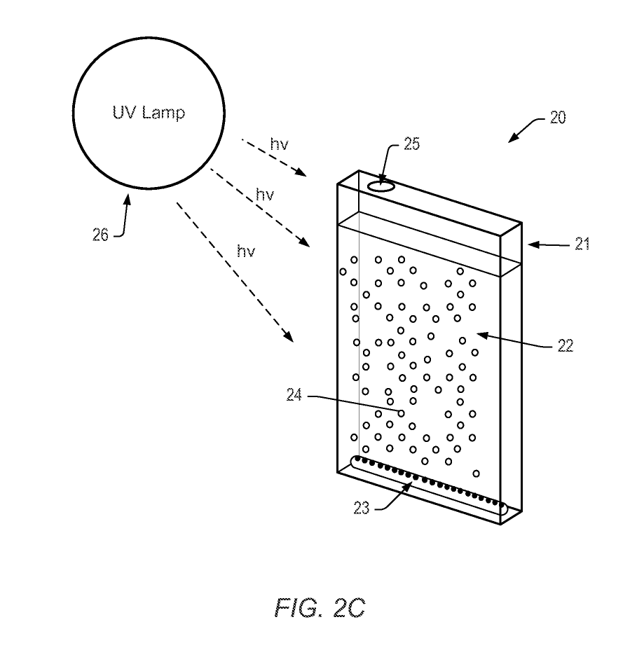

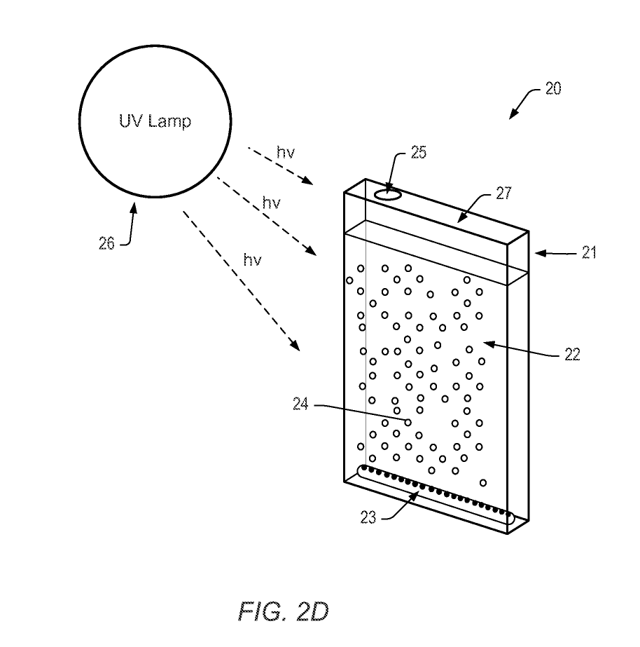

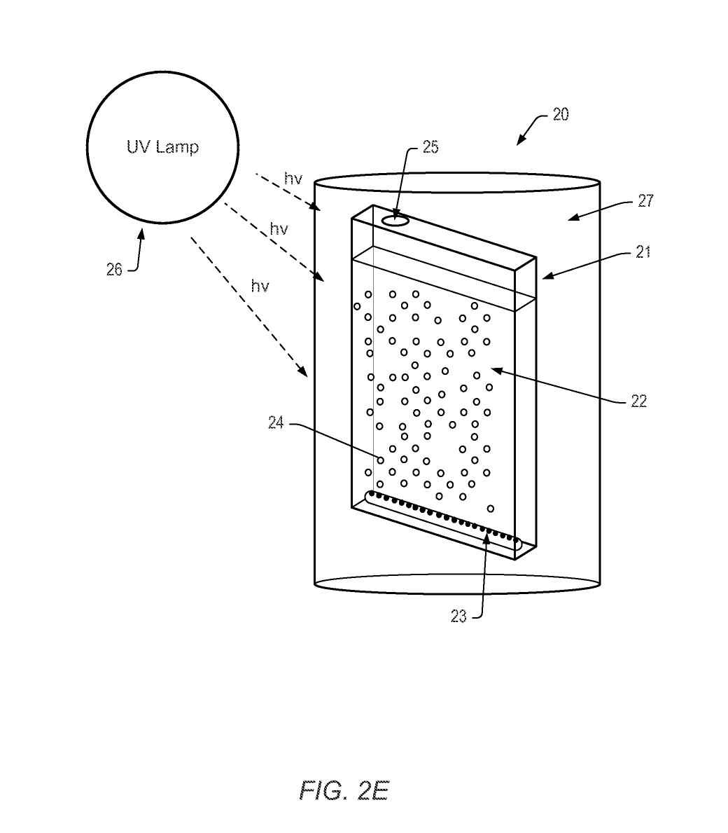

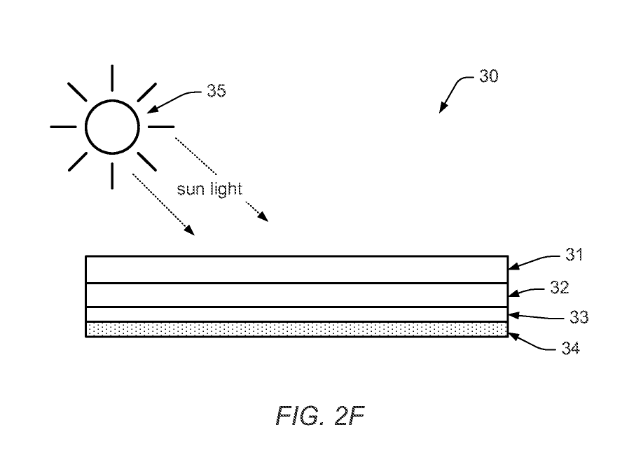

A unique aspect of the present invention is that the polymeric matrices can be used as materials in photo-bioreactors to produce phototropic organisms and biomass in a more efficient and robust manner. FIGS. 2(a) and (b) provide a non-limiting illustration of an open-system photo-bioreactor that utilizes a wavelength conversion material of the present invention. FIGS. 2(c)-(f) provide non-limiting illustrations of closed-system photo-bioreactors that utilize a wavelength conversion material of the present invention. As noted above, however, all types of open and closed systems are contemplated as being useful in the context of the present invention (e.g., plate or flatbed photo-bioreactors, tubular photo-bioreactors, bubble column photo-bioreactors, foil photo-bioreactors, open pond or race-way photo-bioreactors, etc.). Further, the wavelength conversion materials can be used in each of these non-limiting embodiments to convert light from a less-efficient wavelength to a more efficient wavelength that is more readily useable by the phototropic organisms. The wavelength-conversion material is capable of absorbing light comprising a wavelength of 280 to 650 nm and emitting the absorbed light at a wavelength of 400 to 800 nm. In particular aspects, the wave-length conversion material is capable of absorbing light comprising a wavelength of 450 to 650 nm and emitting the absorbed light at a wavelength of 550 to 800 nm or is capable of absorbing light comprising a wavelength of 280 to 650 nm and emitting the absorbed light at a wavelength of 400 to 700 nm.

Referring to FIG. 2A, which illustrates a top view of a photo-bioreactor system (10), it is an open-system reactor that includes a continuously looped channel (11) that is configured to contain a liquid medium (14) having the phototropic organism. The liquid medium (14) flows in a counter-clockwise direction via a paddle-wheel (16). The liquid medium (14) can include a phototropic organism such as algae or cyanobacteria or combinations thereof, water, carbon-dioxide, and additional nutrients for the algae or cyanobacteria. An inlet (12) can be used to provide additional water, nutrients, and phototropic organisms. Inlet (13) can be used to provide a source of carbon dioxide. In particular aspects, the source of carbon dioxide can be obtained by locating the system (10) near a conventional fossil fueled power plant so as to use the carbon dioxide from the smokestacks, thereby providing a carbon dioxide source for algae or cyanobacteria production while reducing carbon dioxide pollution at the same time. An outlet (15) can be used to harvest the biomass from the system (10). A wavelength conversion material in the form of a thin transparent or translucent sheet or film (17) is placed over the channel (11) such that a light source (18) comes into contact with the sheet (17), thereby initiating the conversion of the light to a wavelength that is more efficiently used by the phototropic organisms in the liquid medium (14). FIG. 2B provides a side view of the system (10), which further illustrates the sheet's (17) proximity to the channel (11) and the liquid medium (14) with said channel (11). The thickness of the sheet or film (17) can be varied as desired. In particular aspects, the thickness ranges from 10 to 500 μm or of 0.5 to 3 mm.

FIG. 2C provides an illustration of a closed-system photo-bioreactor (20) that is shaped like a flat plate. The walls (21) of the photo-bioreactor are made of the wave-length conversion material of the present invention. The liquid medium (14) can include a phototropic organism such as algae or cyanobacteria or combinations thereof, water, carbon-dioxide, and additional nutrients for the algae or cyanobacteria. An inlet (23) can be used to provide a source of carbon dioxide (24). An outlet (25) can be used to remove excess carbon dioxide (24) or to harvest the produced biomass. In this embodiment, an artificial light source (26) is used. However, natural sunlight can be used in lieu of or in addition to said artificial light (26). In a further embodiment, the walls (21) of the photo-bioreactor may not be made of the wavelength conversion material. Instead, said walls (21) can be coated with a thin film or sheet of the wavelength conversion material (27—illustrated in grey) adhered to the walls (21) (FIG. 2D). The thickness of the sheet or film (27) can be varied as desired. In particular aspects, the thickness ranges from 10 to 500 μm or of 0.5 to 3 mm. In still another embodiment, a thin sheet or film (27) of said wavelength conversion material can be placed between said walls (21) and said light source (26) (FIG. 2E) rather than being adhered to said walls (21). While the thin sheet or film (27) is in the form of a tubular sleeve in FIG. 2E, other shapes are contemplated. Further, the thin sheet or film (27) can be placed between the walls (21) and light source (26) such that the film (27) does not encompass the bioreactor (20) like a tubular sheet but rather is simply there between said walls (21) and light source (26).

FIG. 2F illustrates a stack (30) of flatbed photo-bioreactors. A first flatbed photo-bioreactor (31) is stacked onto a second flatbed photo-bioreactor (32). The second flatbed photo-bioreactor (or the bottom photo-bioreactor in a stack) includes a wavelength conversion material (33) in the form of a film or sheet adhered to its bottom surface. A reflective backing sheet (34) such as aluminum foil or the like is adhered to the wavelength conversion material (33). This set-up allows for a more efficient use of any unabsorbed light (35). In particular, and in typical stacking modules or systems, the phototropic organisms typically have a more robust growth pattern in the top photo-bioreactor (31) when compared with bottom reactors (32). The reason for this is due to less light (35) being available for said bottom reactors (32). By including a wavelength conversion material (33) in the form of a thin film or sheet, any unabsorbed light will be converted to a more useable wavelength (e.g., 400 to 800 nm), and then the reflective backing (33) will reflect the converted light back into the bottom stack (32). The thickness of the sheet or film (33) can be varied as desired. In particular aspects, the thickness ranges from 10 to 500 μm or of 0.5 to 3 mm.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Polymeric Matrices

Table 2 provides the compositional characteristics of various types of prepared polymeric matrices/films:

TABLE 2

| Sample | HP0322N | 518N | NLR | Tinuvin 783 |
|---|---|---|---|---|
| ID | (kg) | (kg) | (g) | (g) |
| B | 20 | 0 | 0 | 0 |
| C | 20 | 0 | 0 | 200 |
| D | 14 | 6 | 0 | 0 |
| E | 20 | 0 | 50 | 0 |
| F | 20 | 0 | 80 | 0 |
| G | 14 | 6 | 50 | 0 |
| H | 20 | 0 | 50 | 200 |
| I | 20 | 0 | 25 | 0 |

### Example 2

### Process for Making Polymer Matrices

The following process was used to prepare the polymeric matrices of Table 2: Mixed polymer with organic fluorescent dye in a zip-lock polyethylene bag and shake vigorously for about 3-4 minutes. Extruded mixture with a Clear ZSK30 twin screw extruder under the following conditions in Table 3 to produce polymer pellets in polyethylene:

TABLE 3

| | |
|---|---|
| Feed Zone Temp. (50° C.) | |
| Zone 1 Temp. (130° C.) | Throat/Die Temp. (170° C.) |
| Zone 2 Temp. (140° C.) | Screw Speed (rpm of 250) |
| Zone 3 Temp. (150° C.) | Temp. of Melt (170° C.) |
| Zone 4 Temp. (160° C.) | Torque (Nm) (22-23) |

Subsequently, the pellets were subjected to molding using an LTM-Demag from L&T Plastics Machinery Ltd. under the following conditions in Table 4:

TABLE 4

| Extruder | Units | Kiefel Blown Film |
|---|---|---|
| Sample ID-LDPE HP0322N | | Set |
| RPM | RPM | 60 |
| Hauloff speed | m/min | 18 |
| Extr. Screw Bushing Temp | (° C.) | 175 |
| (1) | | |
| Extr. Screw Bushing Temp | (° C.) | 180 |
| (2) | | |
| Extr. Screen Changer Temp | (° C.) | 185 |
| Blow Head Zone Temp (1) | (° C.) | 190 |
| Blow Head Zone Temp (2) | (° C.) | 190 |
| Blow Head Zone Temp (3) | (° C.) | 190 |
| Blow Head Zone Temp (4) | (° C.) | 190 |
| Air Ring Temp | (° C.) | 11 |
| Blower Speed Cooling Air | (%) | 42 |
| Die Diameter | Mm | 150 |
| Die Gap | mm | 2.3 |
| Screw Type | | HDPE |

### Example 3

### Absorption/Emission Data

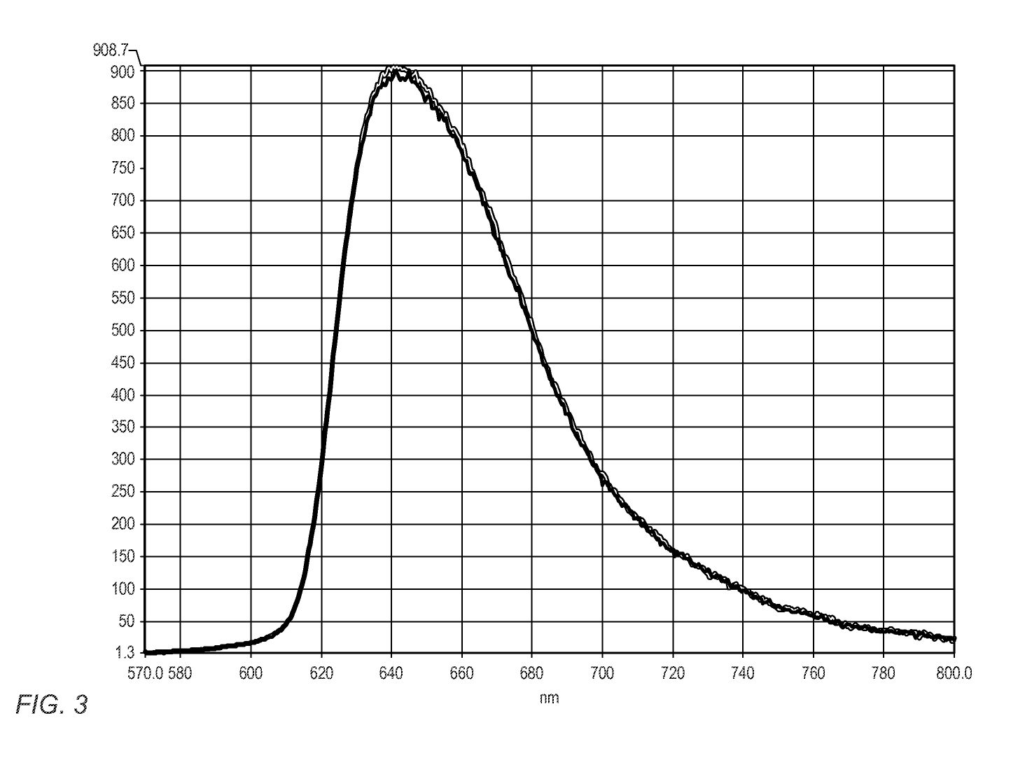

FIG. 3 provides surface emission of films having lumogen red (LR) derivatives incorporated into an LDPE polymer matrix. The light transmission through the film is at different spectral ranges, as indicated in Table 5. These data confirm an increase in the light in the red region in dye incorporated LDPE films.

TABLE 5

| | | | Far | | | |
|---|---|---|---|---|---|---|
| | Blue B | Red R | Red FR | NIR | | |
| Sample | 400-500 | 600-700 | 700-800 | 700-2500 | B/R | R/FR |
| ID | nm | nm | nm | nm | ratio | ratio |
| B | 90.34 | 91.52 | 91.98 | 89.67 | 0.99 | 0.99 |
| C | 90.63 | 91.40 | 91.84 | 90.26 | 0.99 | 1.00 |
| D | 90.35 | 91.24 | 91.57 | 89.90 | 0.99 | 1.00 |
| E | 67.29 | 102.27 | 94.87 | 90.38 | 0.99 | 1.08 |
| F | 62.47 | 102.76 | 95.24 | 90.61 | 0.99 | 1.08 |
| G | 60.21 | 105.09 | 95.42 | 90.14 | 0.99 | 1.10 |
| H | 67.44 | 102.57 | 94.25 | 90.39 | 0.99 | 1.09 |
| I | 76.15 | 100.47 | 93.64 | 90.56 | 0.99 | 1.07 |

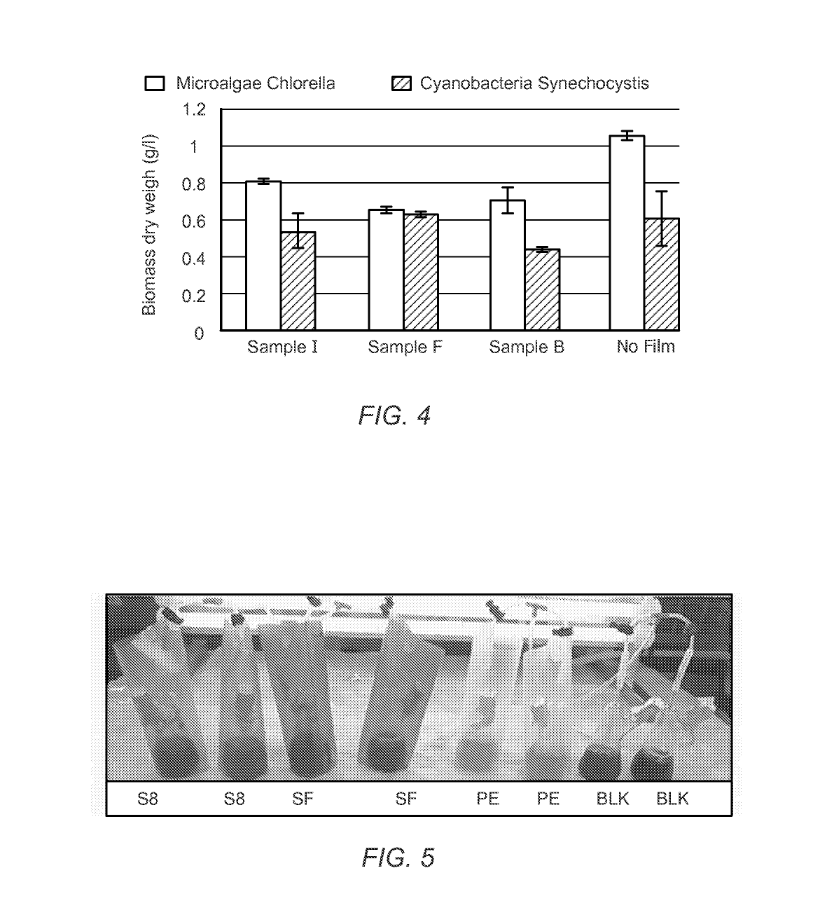

In an effort to characterize the growth of phototropic organisms, Cyanobacteria synchocystis PCC6803 and Microalgae chlorella were used. These organisms were grown indoors under simulated light conditions with the bioreactors covered with sleeves made of dye incorporated low density polyethylene (LDPE) film (FIG. 4). The biomass was dry weight was measured at the end of each experiment and was compared with blank polyethylene (PE) as well as without film. It was observed that the biomass of microalgae covered with film formulation of Sample I (0.81 g/l) is higher than that in PE (0.71 g/l) by 14%. The biomass of cyanobacteria in Sample F (0.54 g/l) is higher than that in PE (0.44 g/l) by 23% (FIG. 5).

The particular growth conditions included: medium of BG-11; light of 50 μE m⁻² s⁻¹. When cultured OD reached 0.1 on day 2, all the cultures were mixed and equally dispensed back into the flasks covered by the NLR PE films (S8: low NLR con; SF: high NLR con). Daily culture optical density was measured, and final cell density was measured as biomass dry weight.

### Absorption/Emission Data of Polyethylene Extruded Films with Perylene Dyes of the Present Invention

Performance of Polyethylene Films Extruded with Luminescent (NRL) Dyes of the Present Invention in Sunlight.

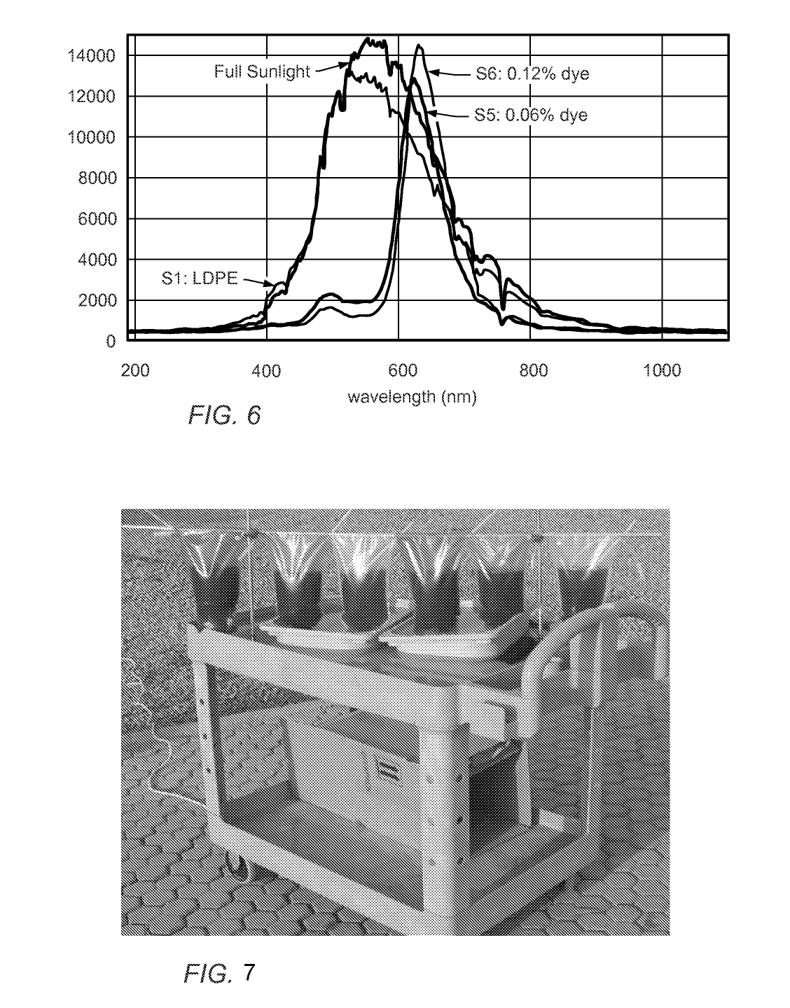

PE beads doped with nonyl phenyl containing lumogen red (NRL) dyes of the present invention having the structure depicted in Formula II were prepared using the procedure outlined in Example 2. PE beads and PE doped beads were extruded into 0.1 mm thick polyethylene rolls having a width of 38 cm. The solar spectra (natural or shifted by the red films) that passed through the NLR films and a control PE film outdoors at noon on a sunny day were measured by a spectrometer HR2000+ES (OceanOptics). The solar intensities were measured by light meter LI-250A (LI-COR). Table 6 lists the modification of sunlight by the extruded PE films containing nonyl perylene luminescent dye (PE+NRL) of the present invention and the light absorbed by photosynthetic pigments, algae/cyanobacteria (Chl a), algae (Chl b), and cyanobacteria (phycocyanin) at the listed wavelengths. FIG. 6 depicts a relative spectra of sunlight measured for PE films containing two concentrations of NLR (0.06% NRL (PE+S5) and 0.12% NLR (PE+S6). The full sunlight spectrum is indicated by the colorful bell; green bell, sunlight altered by PE film; blue bell, sunlight altered by S5 (PE with 0.06% NLR); red bell, sunlight altered by S6 (PE with 0.12% NLR). As shown in Table 6 and FIG. 6, the NLR films shifted the sunlight spectrum towards red. Furthermore, from the data it was concluded that the NLR films reduced blue light (e.g., 67% of blue in sunlight by S6), the NLR films increased red light (e.g., 100% of red in sunlight by S6) and the NLR films also increased the lights absorbed photosynthetic pigments (Chl a, Chl b and phycocyanin).

TABLE 6

| | | S5 | S6 |
|---|---|---|---|
| Intensity change % of | S1 | PE+ | PE+ |
| sunlight chromatography | Blank PE | 0.06% NLR | 0.12% NLR |
| Blue 450-495 nm | 1.9 | −55 | −67 |
| Green 495-570 nm | −10 | −72 | −82 |
| Yellow 570-590 nm | −17 | −58 | −71 |
| Orange 590-620 nm | −18 | 21.4 | 0.1 |
| Red 620-700 nm | −18 | 73.7 | 100 |
| Chl a 650-700 nm | −18 | 27.7 | 51.1 |
| (algae/cyanobacteria) | | | |
| Chl b 600-650 nm (algae) | −18 | 75 | 77.6 |
| Phycocyanin | −18 | 72.7 | 92.8 |
| 610-700 nm (cyanobacteria) | | | |

### Example 5

### Growth of Cyanobacterium Under Outdoor Conditions in PE Bags Doped with Dyes of the Present Invention

Growth of Synechococcus leopoliensis B625 Under Outdoor Conditions with Nonyl Phenyl Perylene Dyes Doped PE Film.

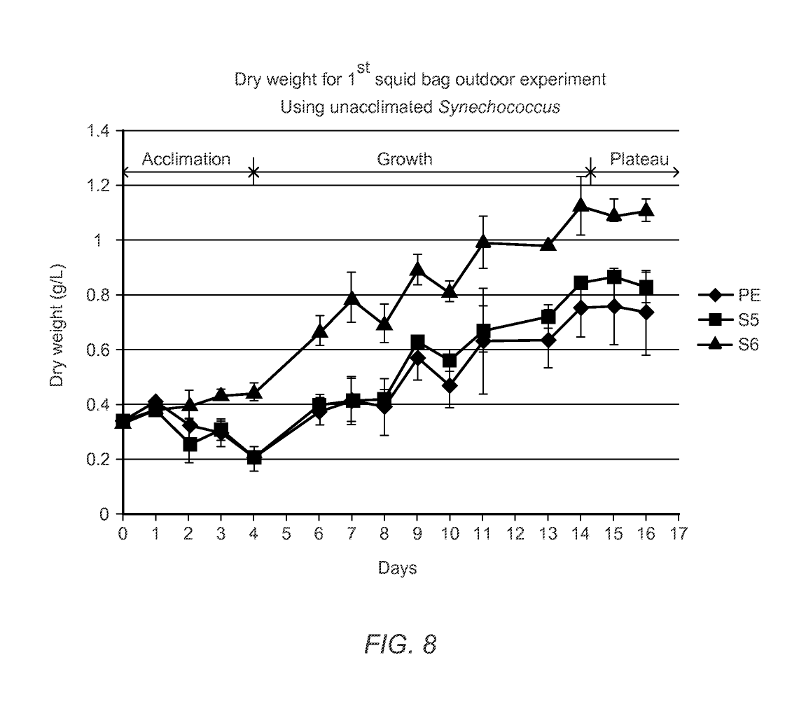

The cyanobacterium Synechococcus leopoliensis B625 (UTEX #B625) was grown in freshwater BG-11 media outdoors where natural sunlight was reached a maximum of around 2000 μE m⁻² s⁻¹ (August). The cyanobacterium was grown in six aerated 1.3-liter bag reactors as duplicates with 0.12% (S6), 0.06% (S5) and 0% (PE) NLR dye of the present invention added to PE film over a 16 day period. Temperature control was maintained by using a water sink, which kept the culture temperature between 36-39° C. (FIG. 7). Cell samples were taken in the morning at 10 am. FIG. 8 is a graph of days of growth of acclimated Synechococcus leopoliensis B625 dry weight in grams per liter. As depicted in FIG. 8, a 4-5 day acclimatization period was experienced before cell density increased significantly from the point of inoculation, which was more proficient in PE bags with the highest NRL dye concentration (S6) of the present invention. The dry weight increased from 0.3 g/l to 1.1 g/l in 16 days compared with from 0.3 to 0.75 g/l for the control culture (PE). Biomass production rates were found to be 0.11, 0.10, and 0.07 g/l/day for S5, S6 and PE cultures, respectively.

### Example 6

### Growth of Cyanobacterium with Nitrogen in PE Bags Doped with Dyes of the Present Invention

Growth of Synechococcus sp. PCC7002 with Urea as a Nitrogen Source in NLR Doped PE Film.

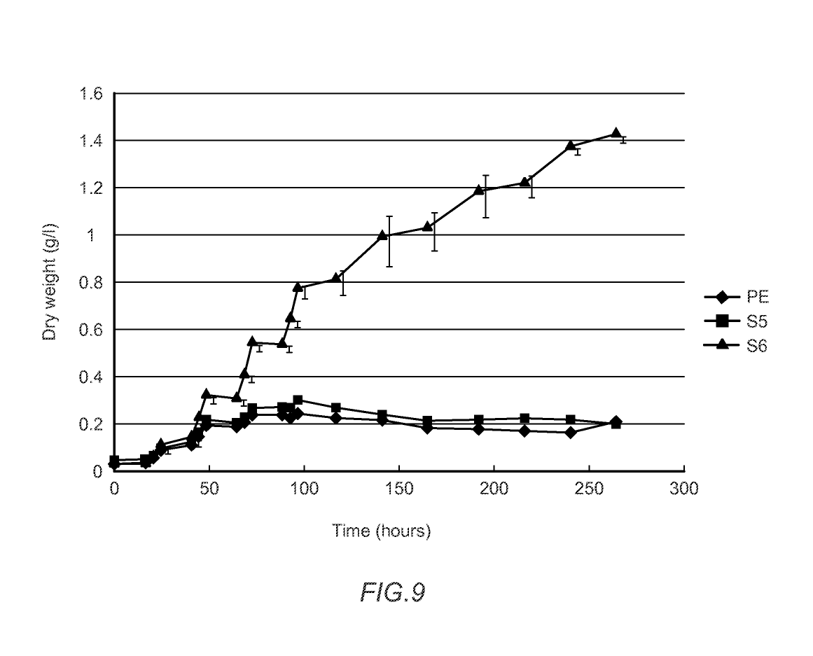

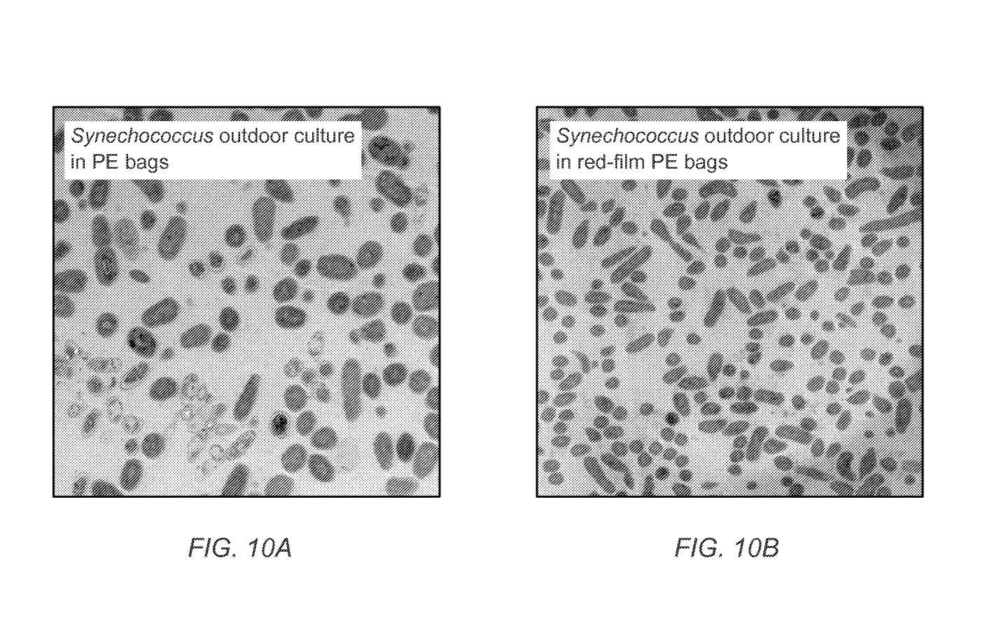

The cyanobacterium Synechococcus sp. PCC7002 (7002 hereafter) was grown in sea-water MN media supplemented with 0.6 g/l urea outdoors when the natural sunlight reached 1,400 μE m-2 s-1 (November). The cyanobacterial cultures were grown in nine aerated 1.3-liter bag reactors (PE, S5, and S6) as triplicates and were maintained at 39° C., without the need for the cooling using the setup depicted in FIG. 7. Cell samples were taken three times during the day. FIG. 9 is a graph of time versus dry weight for the cyanobacterium dry weight in grams per liter. As depicted in FIG. 9, the culture grown in PE film bags containing NRL dye at 0.12% was able to acclimatize and reached a biomass of 1.4 g/L. The other samples were not able to acclimatize. The cell cultures were analyzed using transmission electron microscopy (TEM). FIGS. 10A and 10B depict TEM images of cell controls for the control sample (PE, FIG. 10A) and the bag doped with 0.12 wt. % of NRL of the present invention (S6, FIG. 10B). The cells grown in the bags made with 0.12% perylene-based dyes of the present invention had a better cell integrity than the cells grown in undoped PE bags (control sample) as the number of atrophied cells per field of view is greater for the control PE bags. Thus, bags of the present invention provide a protective role for cultures grown outdoors.

1. A photo-bioreactor configured for growing phototrophic algae and/or cyanobacteria organisms, comprising a wavelength conversion material comprising an organic fluorescent dye and a polymeric matrix, wherein the organic fluorescent dye is solubilized in the polymeric matrix, and wherein the wavelength-conversion material is capable of absorbing light comprising a wavelength of 400 to 650 nm and emitting the absorbed light at a wavelength of 500 to 800 nm across the entire surface of the wavelength conversion material.

2. The photo-bioreactor of claim 1, wherein the material is capable of absorbing light comprising a wavelength of 450 to 650 nm and emitting the absorbed light at a wavelength of 550 to 800 nm.

3. (canceled)

4. The photo-bioreactor of claim 1, wherein the organic fluorescent dye is a perylene containing compound.

5. The photo-bioreactor of claim 4, wherein the perylene containing compound is a perylene di-imide.

6. The photo-bioreactor of claim 5, wherein the perylene di-imide has a structure of: 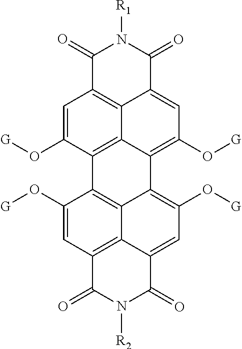
wherein
R₁ and R₂ are each independently selected from branched C₆-C₁₈ alkyl and phenyl which is disubstituted by C₁-C₅ alkyl; and
G is independently selected from 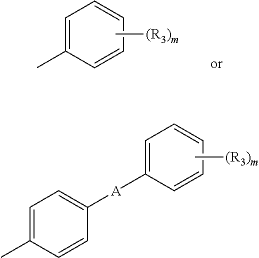
wherein
R₃ is independently selected from hydrogen, C₈-C₁₂ alkyl and halogen;
m represents the number of substituents and is an integer from 0 to 5;
R₄ is independently selected from hydrogen, C₁-C₁₂ alkyl, C₆-C₂₀ aromatic, and C₆-C₂₀ cycloalkyl;
n represents the number of substituents and is an integer from 0 to 5; and
A is selected from a bond, C₁-C₁₂ alkyl, C₆-C₂₀ aromatic, and C₆-C₂₀ cycloalkyl.

7. The photo-bioreactor of claim 6, wherein the perylene di-imide has a structure of:

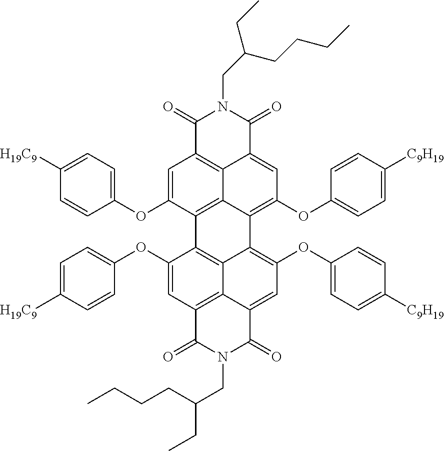

8. The photo-bioreactor of claim 4, wherein the perylene containing compound has a structure of: 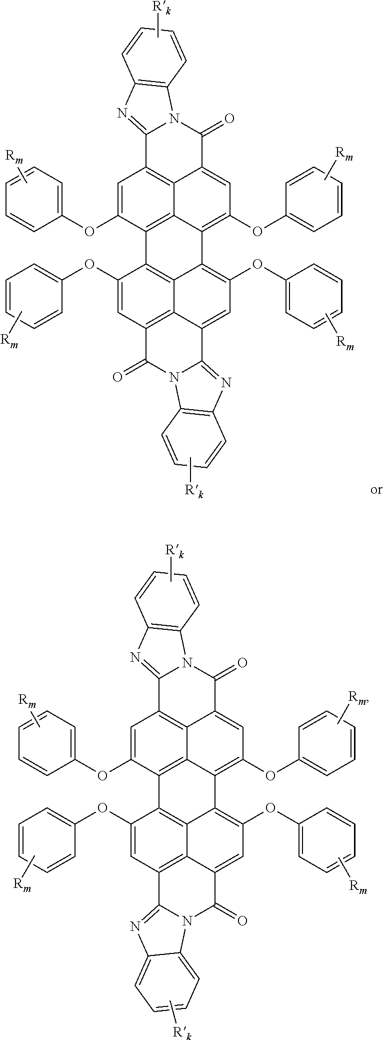
wherein
R and R′ are each independently selected from C₈-C₁₈ alkyl, substituted C₈-C₁₈ alkyl, C₈′ C₁₈ alkoxy, substituted C₈-C₁₈ alkoxy, and halogen;
m represents the number of R substituents on each phenoxy ring, wherein each m is independently an integer from 0 to 5; and
k represents the number of R′ substituents on each benzimidazole group, wherein each k is independently an integer from 0 to 4.

9. The photo-bioreactor of claim 1, wherein the material is configured to be placed between a light source and a plurality of at least one phototrophic organism.

10. The photo-bioreactor of claim 9, wherein the plurality of the at least one phototrophic organism is comprised in a liquid medium or is supported on a substrate such as a solid substrate or a semi-solid or gel substrate.

11. The photo-bioreactor of claim 10, wherein the wavelength-conversion material is configured to form at least a portion of a container that is configured to hold the liquid medium comprising the plurality of the at least one phototrophic organism.

12. The photo-bioreactor of claim 10, wherein the plurality of the at least one phototrophic organism is supported on a substrate and is capable of forming a biofilm on said substrate.

13. The photo-bioreactor of claim 1, wherein the phototrophic organisms comprise algae or cyanobacteria.

14. (canceled)

15. (canceled)

16. The photo-bioreactor of claim 1, wherein the wavelength-conversion material is transparent or translucent.

17. (canceled)

18. (canceled)

19. The photo-bioreactor of claim 1, wherein the wavelength-conversion material is a film having a thickness of 10 to 500 μm or of 0.5 to 3 mm.

20. (canceled)

21. The photo-bioreactor of claim 1, wherein the organic fluorescent dye is thermally stable at a temperature from 200 to 350° C.

22.-23. (canceled)

24. The photo-bioreactor of claim 23, wherein the polymeric matrix comprises a polycarbonate or a polyolefin or a combination thereof.

25. The photo-bioreactor of claim 23, wherein the polymeric matrix comprises a polyolefin and wherein the polyolefin is a polyethylene or polypropylene polymer.

26-29. (canceled)

30. The photo-bioreactor of claim 29, wherein the wherein the wavelength conversion material further comprises an additive having a structure of: 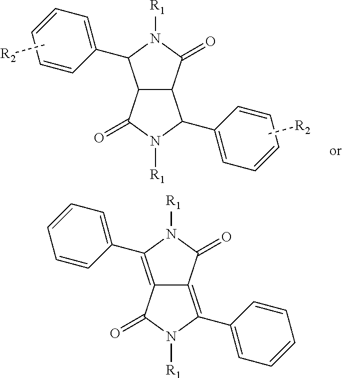
wherein R₁ and R₂ are each individually H, CH₃, CH₂H₅, 2-ethylhexyl, an amine, or a halogen.

31.-43. (canceled)

44. The photo bioreactor of claim 1, wherein the wavelength conversion material further comprises a diffuser, wherein the diffuser comprises cross-linked siloxane particles.

45-58. (canceled)

59. A method of growing a phototrophic organism comprising:
(a) obtaining a plurality of at least one phototrophic organism;
(b) converting light comprising a wavelength of 280 400 to 650 nm into light comprising a wavelength of 400 500 to 800 nm with any one of the wavelength-conversion materials of claims 1 to 43 the photo-bioreactor of claim 1; and
(c) subjecting the plurality of the at least one phototrophic organism to the converted light.

60-71. (canceled)
